(19) United States
(12) Patent Application Publication
(10) Pub. No.: US 2012/0258217 A1
(43) Pub. Date: Oct. 11, 2012

(54) METHOD OF PROCESSING A JUICE AND/OR SODA PRODUCT

(75) Inventor: Matthias Weinzierl, Eching (DE)

(73) Assignee: KRONES AG, Neutraubling (DE)

(21) Appl. No.: 13/433,360

(22) Filed: Mar. 29, 2012

(30) Foreign Application Priority Data
Apr. 1, 2011 (DE) 10 2011 006 655.1

(51) Int. Cl. G01N 33/14; B65B 3/04; A23L 2/58; A23L 2/02; A23L 2/38

(52) U.S. Cl. 426/232; 426/231; 99/275; 141/83

(57) ABSTRACT

A method of processing a juice and/or soda product in a beverage processing plant, where the juice and/or soda product is obtained from one or several starting products, including determining the color and turbidity of at least one starting product of a juice and/or soda product, and/or the color and turbidity of the obtained juice and/or soda product by means of at least one sensor of the beverage processing plant, comparing the determined color and turbidity with a stored color and a stored turbidity of at least one starting product of a predetermined juice and/or soda product, and/or with a stored color and a stored turbidity of the predetermined juice and/or soda product, and operating the beverage processing plant based on the result of this comparison.

### CROSS-REFERENCE TO RELATED APPLICATION

The present application claims the benefit of priority of German Application No. 102011006655.1, filed Apr. 1, 2011. The entire text of the priority application is incorporated herein by reference in its entirety.

### FIELD OF THE DISCLOSURE

The present disclosure relates to a method of processing, in particular manufacturing and/or filling, a juice and/or soda product in a beverage processing plant.

### BACKGROUND

In the beverage industry, most diverse juice and/or soda products are often processed successively in the same beverage processing plant. In a format change to a new product, it can occur that some residues of the previously processed juice and/or soda product are still present in the beverage processing plant. This can have a negative effect on the quality of the new juice and/or soda product to be processed.

It is also conceivable that after a format change, the correct ingredients or starting products for the new juice and/or soda product are not provided. For example, an operator can introduce a wrong starting product into the beverage processing plant which, however, is optically similar to the desired starting product. If this error is recognized too late, this can lead to cost-intensive losses of production.

### SUMMARY OF THE DISCLOSURE

It is one aspect of the present disclosure to provide a method of processing, in particular manufacturing and/or filling, a juice and/or soda product in a beverage processing plant which permits a reliable processing of the juice and/or soda product.

The disclosure provides a method of processing, in particular manufacturing and/or filling, a juice and/or soda product in a beverage processing plant, wherein the juice and/or soda product is obtained from one or several starting products, including the steps of:

determining the color and turbidity of at least one starting product of a juice and/or soda product and/or the color and turbidity of the obtained juice and/or soda product by means of at least one sensor of the beverage processing plant,

comparing the determined color and turbidity with a stored color and a stored turbidity of at least one starting product of a predetermined juice and/or soda product, and/or with a stored color and a stored turbidity of the predetermined juice and/or soda product, and

operating the beverage processing plant based on the result of this comparison.

By measuring the color and the turbidity of at least one starting product of a juice and/or soda product and/or the final juice and/or soda product and comparing it with the corresponding stored values of a predetermined or desired juice and/or soda product, a wrong or qualitatively not sufficient starting product or deviations of the obtained juice and/or soda product from desired specifications can be detected. By this, a more reliable operation of the beverage processing plant can be achieved. In particular, quality control becomes possible thereby.

Processing a juice and/or soda product can here mean any treatment steps including the juice and/or soda product or individual starting products of it. For example, the manufacture and/or filling of a juice and/or soda product can be referred to as processing.

Juice product can here in particular be understood as a beverage comprising at least one ingredient based on a plant. In particular, at least one starting product of the juice product can be a fruit and/or vegetable juice. Syrup can also be referred to as juice product.

A non-alcoholic beverage on the basis of water can be referred to as soda product, which, apart from water, also comprises flavoring preparations, artificial and/or natural flavors, sugar and/or sweetener. A soda product can also comprise a fruit juice proportion and/or a vegetable juice proportion. The fruit juice proportion and/or the vegetable juice proportion of a soda product, however, can be lower than the juice proportion and/or the vegetable juice proportion of a juice product.

The juice and/or soda product can be mixed with carbon dioxide. Thereby, a carbonized soft drink (CSD) can be obtained.

Starting products can be understood here as components or ingredients from which the final juice and/or soda product is obtained, for example by mixing. In other words, ingredients can be understood as starting products from which the juice and/or soda product can be obtained in the beverage processing plant. As starting products, in particular water, fruit and/or vegetable juices and/or milk are possible.

If the juice and/or soda product is obtained from one, in particular precisely one starting product, the juice and/or soda product can also correspond to the starting product.

Turbidity can be understood as a weakening of the passing luminous radiation and/or a diffusion of the luminous radiation due to particles suspended in the liquid.

The comparison step can be carried out by a control element of the beverage processing plant. The control element can be, for example, a programmable logic controller (PLC) and/or a computer. The color and turbidity of at least one starting product of a predetermined juice and/or soda product and/or the predetermined juice and/or soda product can be stored in a storage element, in particular of the control element.

Processing can be the manufacture of a juice and/or soda product. In other words, the method can comprise a manufacture of the juice and/or soda product. In particular, the method can comprise a manufacture of the juice and/or soda product, manufacture comprising mixing at least two starting products and the determination of the color and turbidity being carried out before, during and/or after mixing.

If the determination of the color and turbidity is carried out before mixing, one can examine, for example, whether the correct starting products are provided for the desired juice and/or soda product. If determination is carried out during mixing, the correct mixing ratio can be checked. If determination is carried out after filling, one can check, for example, whether the produced juice and/or soda product corresponds to the specifications of a recipe taken as a basis.

Processing can be the filling of a juice and/or soda product. This means, the method can comprise a filling of the juice and/or soda product, the determination of the color and turbidity being carried out before and/or during filling.

The comparison step can in particular comprise a detection of the at least one starting product of the juice and/or soda product. In other words, at least one starting product of a juice and/or soda product can be detected or identified, in particular uniquely, on the basis of the determined color and turbidity.

The comparison step can also include a detection of the juice and/or soda product, in particular based on the determined color and turbidity.

The operation of the beverage processing plant based on the result of the comparison can comprise stopping, changing the operating mode, and/or emitting a warning signal. If, for example, a wrong starting product is detected, the beverage processing plant can be stopped, and/or the emission of a warning signal can be caused. This can be done, for example, by a control element of the beverage processing plant.

The warning signal can correspond to an optical and/or an acoustic warning signal. The warning signal can comprise an error message.

A predetermined selection or adjustment of operating parameter values of the beverage processing plant can be referred to as an operating mode. In other words, the beverage processing plant can carry out predetermined processing steps in a predetermined manner in a selected operating mode. Depending on the operating mode, predetermined processing steps may not be carried out. For example, an operating mode can correspond to a stand-by mode, a filling mode or a discharge mode.

In the stand-by mode, processing functions of the plant can be temporarily deactivated, however without completely shutting down the beverage processing plant. By this, the processing functions can be reactivated more quickly if required. In the filling mode, the juice and/or soda product can be processed, in particular filled. In the discharge mode, a starting product and/or a juice and/or soda product can be discharged or pushed out of the beverage processing plant, for example in case of a format change to a new juice and/or soda product to be processed. For this, for example one or several new starting products can be introduced into the beverage processing plant.

A change of the operating mode can be understood as a change between two different operating modes.

In the discharge mode, mixing phases can be determined in particular. Mixing phases can be understood as mixtures between one or several starting products and/or a juice and/or soda product before a format or product change, and those after the format or product change. As soon as it is detected, for example, that the previously processed juice and/or soda product is essentially no longer present in the beverage processing plant, the beverage processing plant can change, for example, to a filling mode.

The steps of the above mentioned processes can be carried out in particular several times, in particular periodically or after predetermined time intervals. By this, an online quality control of the beverage processing plant can be achieved, that means a control of the beverage processing plant during production. For example, the steps can be carried out at an interval of 1 minute. However, an interval of about 0.1 to maximally 5 seconds can also be used, in particular to ensure operating reliability online. Ideally, the interval can be adapted to the cycle time of the control, for example 8 measured values per second. Thus, any change can be directly detected without any loss. With very large flow rates, an even smaller interval can make sense to minimize product losses.

The comparison can in particular comprise the determination whether the determined color and turbidity is in conformity with the stored color and the stored turbidity according to a predetermined criterion. The predetermined criterion can specify predetermined tolerance limits. By this, the desired quality or reliability to be achieved can be flexibly adjusted. By this, one can also take into account errors of measurement in the determination of the color and turbidity.

The operation of the beverage processing plant based on the result of the comparison can comprise a change or adjustment of at least one processing parameter of the beverage processing plant based on the result of the comparison. For example, a mixing ratio in the mixing of several starting products can be changed or adjusted based on the result of the comparison.

In addition to the color and turbidity, one or several further properties of the at least one starting product and/or the juice and/or soda product can also be determined and compared with the corresponding stored property of at least one starting product of a predetermined juice and/or soda product and/or the at least one predetermined juice and/or soda product. By this, the reliability of the method can be further increased. Exemplary further properties can be electric conductivity, temperature and/or density.

The disclosure moreover provides a beverage processing plant for processing, in particular manufacturing and/or filling, a juice and/or soda product, wherein the juice and/or soda product is obtained from one or several starting products, including:

at least one sensor for determining the color and turbidity of at least one starting product of a juice and/or soda product and/or the color and turbidity of the obtained juice and/or soda product,

a storage element in which the color and turbidity of at least one starting product of at least one predetermined juice and/or soda product and/or the color and turbidity of the at least one predetermined juice and/or soda product are stored, and

a control element (14) which is configured and/or embodied such that

a color and turbidity determined by the at least one sensor is compared with the stored color and the stored turbidity of at least one starting product of at least one predetermined juice and/or soda product, and/or the color and turbidity of the at least one predetermined juice and/or soda product, and

the beverage processing plant is operated based on the result of this comparison.

In other words, the beverage processing plant can be designed and/or configured such that it can carry out an above described method of processing a juice and/or soda product. In particular, the control element can be configured and/or embodied such that it carries out an above described method.

The control element can be, for example, a programmable logic controller (PLC) and/or a computer. The storage element can be part of the control element. Thereby, the color and turbidity of a high number of starting products and/or juice and/or soda products can be stored in the storage element.

The beverage processing plant can moreover comprise a filling device for filling the juice and/or soda product, in particular wherein the sensor is arranged upstream of and/or in the filling means.

The beverage processing plant can moreover comprise a mixing device in which a juice and/or soda product is produced by mixing at least two starting products. In this case, the sensor can be arranged upstream of, in and/or downstream of the mixing device. Several sensors can also be arranged in the beverage processing plant, in particular at different positions. By this, quality control at different positions of the beverage processing plant is possible.

The beverage processing plant can in particular also comprise one or several sensors for determining a further property of at least one starting product and/or the juice and/or soda product. In particular, the beverage processing plant can comprise at least one sensor for determining the electric conductivity, the temperature and/or the density of the at least one starting product and/or the juice and/or soda product.

### BRIEF DESCRIPTION OF THE DRAWINGS

Further advantages and features of the disclosure will be described below with reference to the exemplary figures. In the drawings:

FIG. 1 shows a mixing device of an exemplary beverage processing plant;

FIG. 2 shows a filling device of an exemplary beverage processing plant; and

FIG. 3 shows an illustration of an exemplary method of processing a juice and/or soda product in the form of a flow chart.

### DETAILED DESCRIPTION OF THE PREFERRED EMBODIMENT

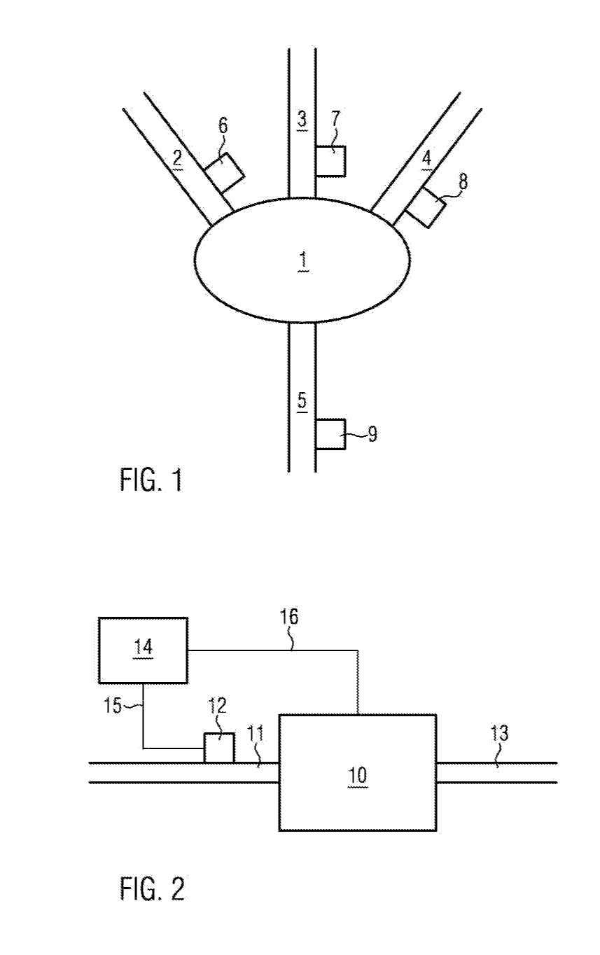

In FIG. 1, an exemplary mixing device of a beverage processing plant is schematically represented. By means of this mixing device, a juice and/or soda product can be produced by mixing three starting products. For example, a fruit juice drink can be produced by mixing three different fruit juices, for example strawberry juice, orange juice and banana juice.

Correspondingly, three supply lines 2, 3 and 4 lead into the mixing device 1, each being provided for one starting product of the juice and/or soda product. Supply line 2 can be provided, for example, for strawberry juice, supply line 3 for orange juice, and supply line 4 for banana juice. The mixing device 1 mixes these starting products or components according to a predetermined mixing ratio. The mixed or obtained juice and/or soda product is discharged from the mixing device 1 via the discharge line 5.

In this example, a sensor 6, 7, 8 and 9 is provided at each of the supply lines 2, 3 and 4 and at the discharge line 5.

With the sensors 6, 7 and 8, the color and turbidity of the respective starting product of the juice and/or soda product can be determined. The sensor 9 permits to determine the color and turbidity of the final juice and/or soda product, that means the mixture of the three starting products.

Sensors by means of which the color and turbidity of a liquid can be determined are basically known. For example, the Company Mettler Toledo distributes a corresponding sensor with the designation “InPro 8300 RAMS”. Up to now, such sensors have not been used for processing juice and/or soda products as these often comprise variations in color and turbidity due to their composition. However, it surprisingly showed that the determined color and the determined turbidity can also be used for the identification of a juice and/or soda product or a starting product of a juice and/or soda product.

By means of a control element of the beverage processing plant not explicitly shown in FIG. 1, the beverage processing plant can be operated based on a result of a comparison of the color and turbidity determined by the sensors 6, 7, 8 or 9 with a stored color and a stored turbidity of at least one starting product of a predetermined or desired juice and/or soda product, and/or a stored color and a stored turbidity of a predetermined or desired juice and/or soda product. For example, one can detect whether the starting products of the juice and/or soda product introduced into the mixing device correspond to the juice and/or soda product to be produced according to a recipe. So, according to the above example, strawberry juice is to be introduced into the mixing device 1 via supply line 2, orange juice via supply line 3, and banana juice via supply line 4. If at least one of these is not correct, the beverage processing plant can be stopped and/or an alarm or a warning signal can be emitted to an operator.

Via the sensor 9, it can be determined, for example, whether the starting products have been mixed in a correct ratio.

FIG. 2 shows an exemplary filling device of a beverage processing plant. Via an exemplary supply line 11, a juice and/or soda product to be filled can be introduced into the filling device 10. The containers filled with the juice and/or soda product, for example bottles, can then be further transported in the beverage processing plant via a transport medium 13, for example to a packer.

In the region of the supply line 11, a sensor 12 for determining the color and turbidity of the juice and/or soda product to be filled is arranged. Via a data line 15, this sensor 12 is connected to a control element 14 of the beverage processing plant. In the control element 14, the color and turbidity determined by the sensor 12 can be compared with the stored color and turbidity of a predetermined or desired juice and/or soda product. As illustrated above, the beverage processing plant can be stopped, and/or a warning signal can be emitted if it is recognized, based on the comparison, that in the supply line 11, a wrong juice and/or soda product is introduced into the filling device 10.

As an alternative or in addition, the control element 14 can be connected to the filling device via a data line 16. Based on the result of the comparison, then at least one operating setting of the filling device 10 can be changed or adjusted. For example, the filling amount can be selected based on the juice and/or soda product detected by the comparison.

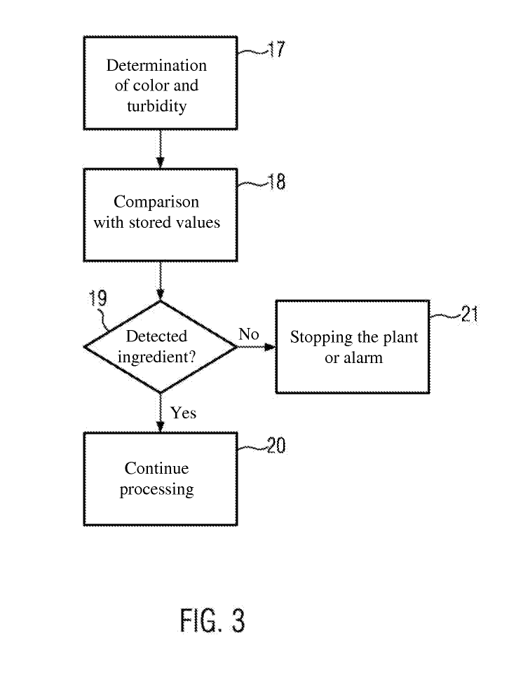

In FIG. 3, an exemplary method of processing a juice and/or soda product in a beverage processing plant is represented with the aid of a flow chart.

In step 17, first the color and turbidity of at least one starting product of a juice and/or soda product and/or the color and turbidity of a juice and/or soda product are determined by means of a sensor of the beverage processing plant. In particular, for all starting products of the juice and/or soda product, the color and turbidity can be determined with one sensor each.

In addition to the color and turbidity, for example conductivity, temperature and/or density can moreover be determined. In step 18, the determined color and turbidity are compared with a color and turbidity stored in a memory with the aid of a control element.

In the memory, in particular the color and the turbidity of all starting products of a desired juice and/or soda product or one to be processed, and/or the color and turbidity of the desired juice and/or soda product itself can be stored.

If the at least one starting product or the juice and/or soda product is detected based on the comparison in step 19, the processing of the juice and/or soda product is continued in step 20. If the at least one starting product or the juice and/or soda product is not detected, in step 21, the beverage processing plant can be stopped and/or an alarm or a warning signal can be emitted, for example to an operator.

It will be understood that features mentioned in the above described embodiments are not restricted to these special combinations and are also possible in any other combinations.

What is claimed is:

1. A method of processing a juice and/or soda product in a beverage processing plant, wherein the juice and/or soda product is obtained from one or several starting products, comprising:
determining via at least one sensor of the beverages processing plant one of the color and turbidity of at least one starting product of a juice and/or soda product, the color and turbidity of the obtained juice and/or soda product by means of at least one sensor of the beverage processing plant, and a combination thereof;
comparing the determined color and turbidity one of with a stored color and a stored turbidity of at least one starting product of a predetermined juice and/or soda product, with a stored color and a stored turbidity of the predetermined juice and/or soda product, and with a combination thereof; and
operating the beverage processing plant based on the result of this comparison.

2. The method according to claim 11, wherein the manufacture comprises mixing at least two starting products, and wherein the determination of the color and turbidity is carried out one of before mixing, during mixing, after mixing, and a combination thereof.

3. The method according to claim 11, wherein the determination of the color and turbidity is carried out one of before filling, during filling, and a combination thereof.

4. The method according to claim 1, wherein the comparison includes detecting the at least one starting product of the juice and/or soda product.

5. The method according to claim 1, wherein the operation of the beverage processing plant based on the result of the comparison includes one of a stop, a change of the operating mode, an emission of a warning signal, and a combination thereof.

6. The method according to claim 1, wherein the steps of the method are carried out several times.

7. The method according to claim 1, wherein the step of comparing is carried out by a control device of the beverage processing plant.

8. The method according to claim 1, wherein the comparison comprises determining whether the determined color and turbidity is in conformity with the stored color and the stored turbidity according to a predetermined criterion.

9. The method according to claim 1, wherein the operation of the beverage processing plant based on the result of the comparison comprises a change or adjustment of at least one processing parameter of the beverage processing plant based on the result of the comparison.

10. A beverage processing plant for processing a juice and/or soda product, wherein the juice and/or soda product is obtained from one or several starting products, comprising:
at least one sensor for determining one of the color and turbidity of at least one starting product of a juice and/or soda product, the color and turbidity of the obtained juice and/or soda product, and a combination thereof;
a storage element for storing one of the color and turbidity of at least one starting product of at least one predetermined juice and/or soda product, the color and turbidity of the at least one predetermined juice and/or soda product, and a combination thereof; and
a control element which is one of configured and embodied such that
a color and turbidity determined by the at least one sensor is compared with one of the stored color and the stored turbidity of at least one starting product of at least one predetermined juice and/or soda product, the color and turbidity of the at least one predetermined juice and/or soda product, and a combination thereof; and
the beverage processing plant is operated based on the result of this comparison.

11. The method according to claim 1, wherein the processing of a juice and/or a soda product comprises one of manufacturing, filling and a combination thereof.

12. The method according to claim 6, wherein the steps of the method are carried out one of periodically and at predetermined time intervals.

13. The beverage processing plant according to claim 10, wherein the processing of a juice and/or a soda product comprises one of manufacturing, filling and a combination thereof.
